(19) United States
(12) Patent Application Publication
(10) Pub. No.: US 2025/0009281 A1
(43) Pub. Date: Jan. 9, 2025

(54) INTRA-OPERATIVE LOWER SPINE MOTOR INTEGRITY MONITORING THROUGH TRANS-SPINAL STIMULATION

(71) Applicant: Alphatec Spine, Inc., Carlsbad, CA (US)

(72) Inventors: Richard Arthur O'Brien, Hunt Valley, MD (US); Robert Gerard Snow, Phoenix, MD (US)

(21) Appl. No.: 18/759,792

(22) Filed: Jun. 28, 2024

Related U.S. Application Data
(60) Provisional application No. 63/511,971, filed on Jul. 5, 2023.

(51) Int. Cl. A61B 5/395; A61B 5/00

(52) U.S. Cl. CPC A61B 5/395; A61B 5/4566; A61B 5/4836

(57) ABSTRACT

Certain aspects of the present disclosure provide techniques for monitoring reflexive motor responses in a patient during a surgery on the patient. A method includes stimulating at least one cathodal electrode over a thoracic spinal region of the back of the patient with at least one anodal electrode over at least one anterior superior iliac crest area of the patient. The method includes detecting, with at least one recording electrode on at least one leg of the patient, one or more resulting muscle response electrical waveforms from one or more muscles of one or more lower extremities of the patient.

### RELATED APPLICATIONS

A claim for priority to the Jul. 5, 2023 filing date of U.S. Provisional Patent Application No. 63/511,971, filed on Jul. 5, 2023, titled INTRA-OPERATIVE LOWER SPINE MOTOR INTEGRITY MONITORING THROUGH TRANS-SPINAL STIMULATION (“the '971 Provisional Application”), is hereby made. The entire disclosure of the '971 Provisional Application is hereby incorporated herein.

### BACKGROUND

### Field of the Disclosure

Aspects of the present disclosure relate to neurophysiological monitoring, and more particularly, to techniques for intra-operative monitoring of the nervous system during surgeries where the nervous system may be at risk.

### Description of Related Art

Certain surgeries, such as lumbar surgery or cervical surgery, risk damage to the patient's nervous system. Lumbar surgery, also known as lumbar spine surgery or lower back surgery, refers to surgical procedures performed on the lumbar region of the spine. The lumbar region is the lower part of the spine, consisting of the five vertebrae (L1 to L5) between the thoracic spine (upper back) and the sacrum (pelvic region). Lumbar surgery is typically considered when conservative treatments such as medication, physical therapy, and lifestyle changes have failed to alleviate severe or chronic back pain, instability, or other symptoms associated with conditions affecting the lumbar spine. Examples of lumbar surgical procedures include disectomy, spinal fusion such as lateral lumbar interbody fusion (LLIF) and transforaminal lumbar interbody fusion (TLIF), laminectomy, foraminotomy, posterior transposas procedures (PTP), and artificial disk replacement surgeries. In order to mitgate the risks of damage during such surgeries, it is helpful to perform neurophysiological monitoring of the motor pathways during such surgeries to detect early changes in neurological dysfunction that if left un-mitigated might lead to transient or permanent injury or dysfunction post operatively.

Such neurophysiological monitoring may include a wide range of techniques including for example short latency Somatosensory Evoked Potentials (SSEP), Trans-Cranial Electrical Motor Evoked Potentials (TceMEP), Electroencephalography (EEG), Hoffman waves (H waves), near nerve stimulation and recording from nerve and/or muscle (triggered EMG), and neuromuscular junction testing for paralytic effect (e.g., Train of Four (TOF) nerve stimulation and muscle recording). Current neurophysiological monitoring approaches, however, have drawbacks including patient movement (e.g., abdominal and axial muscular movement) that can disrupt surgical procedures, being subject to anesthetic effect which can lead to false negative or false positives in detection of changes in neurological dysfunction, use of invasive stimulator electrodes, and limited to intermittent use.

TceMEP is a technique used to assess the integrity of the motor pathways in the brain and spinal cord through the application of electrical stimulation to the motor cortex of the brain, such as through scalp electrodes, while monitoring the electrical responses in the muscles of the body. During a TceMEP procedure, electrical pulses are delivered to the motor cortex through the cranium, which activate the motor neurons responsible for controlling specific muscles. The electrical signals generated by these neurons travel down the spinal cord and peripheral nerves, causing muscle contractions. These muscle contractions produce electrical activity, which can be recorded using electrodes placed near, on or in the muscles. By analyzing the recorded electrical responses, neurophysiologists can evaluate the time it takes for the electrical signals to travel from the motor cortex to the muscles, as well as the amplitude and waveform characteristics of the evoked potentials. Any abnormalities or delays in the response can provide insights into the functional status of the motor pathways and help diagnose conditions such as spinal cord injury. TceMEP helps surgeons avoid damaging critical motor pathways and provides real-time feedback on the functional integrity of these pathways.

TceMEP is often of particular interest to clinical users as it directly measures the integrity of the motor tracts, which is a primary concern. Typically, motor tract recordings are collected on one side and then the other so that individual side testing can be performed as appropriate. Unfortunately, TceMEP has several limitations in both use and interpretation.

One drawback to TceMEP is that it may cause excessive axial movement, which may cause surgical disruption and/or render results difficult to obtain. Another drawback to TceMEP is its sensitivity to inhaled anesthetic agents requiring use of total intravenous anethesia (TIVA). In addition, TceMEP requires careful stimulator electrode placement. Other drawbacks of TceMEP include the need for corkscrew or needle electrodes that can lead to needle sticks or local bruising and bleeding, the occurrence of frequent false positive alerts, and the tendency of the method to activate distal muscles more easily even when proximal muscles may be more of interest. In addition, there is disagreement among neural monitoring experts on appropriate alerting criteria.

For lumbar surgeries, attempts have been made to circumvent some of these issues by using a series of high amperage (e.g., over 450 mA), short-pulsed stimulations through electrodes over the lower back and the abdomen with the goal of directly activating the spinal cord conus or exiting nerve roots. As with TceMEP, however, this technique also suffers from disadvantages, such as surgical disruption due to patient movement. Another disadvantage of this technique is that the high amperage produces a large current field, which is prone to false negative recordings due to over-stimulation and conduction lead (e.g., nerve activation far from the stimulator placement) past the injury site of the nerve or nerve root of interest.

Some techniques stimulate motor function in the lower extremities for non-surgical use, including improving ambulation following spinal cord injury. In these cases, the stimulation is of relatively lower intensity and is meant to depolarize the sensory roots over multiple spinal levels concurrently, leading to a reflex activation of the motor roots. These approaches may aim to activate both sides of the spine simultaneously through anodal electrode placement over the abdomen. Like the TceMEP and high amperage techniques described above, this approach also suffers from considerable abdominal movement. Using this approach in a surgical case would be difficult or impossible as the abdominal electrode placement might interfere with the sterile surgical field. In addition, this technique requires manual activation and searching for an optimum stimulation intensity. Lastly, this technique cannot be applied to the cervical area due to risk of excessive movement and activation of unwanted structures such as respiratory nerves and autonomic nerves and others.

Consequently, there exists a need for further improvements in neurophysiological monitoring that can be performed intra-operatively while overcoming the aforementioned technical challenges and others.

### SUMMARY

One aspect provides a method for monitoring reflexive motor responses in a patient during a surgery on the patient. A method includes stimulating at least one cathodal electrode over a thoracic spinal region of the back of the patient with at least one anodal electrode over at least one anterior superior iliac crest area of the patient. The method includes detecting, with at least one recording electrode on at least one leg of the patient, one or more resulting muscle response electrical waveforms from one or more muscles of one or more lower extremities of the patient.

Other aspects provide: an apparatus operable, configured, or otherwise adapted to perform any one or more of the aforementioned methods and/or those described elsewhere herein; a system for performing the aforementioned methods as well as those described elsewhere herein; a non-transitory, computer-readable media comprising instructions that, when executed by a processor of an apparatus, cause the apparatus to perform the aforementioned methods as well as those described elsewhere herein; a computer program product embodied on a computer-readable storage medium comprising code for performing the aforementioned methods as well as those described elsewhere herein; and/or an apparatus comprising means for performing the aforementioned methods as well as those described elsewhere herein. By way of example, an apparatus may comprise a processing system, a device with a processing system, or processing systems cooperating over one or more networks.

The following description and the appended figures set forth certain features for purposes of illustration.

### BRIEF DESCRIPTION OF DRAWINGS

The appended figures depict certain features of the various aspects described herein and are illustrative and exemplary in nature and not to be considered limiting of the scope of this disclosure. The following detailed description of the illustrative aspects can be better understood when read in conjunction with the following drawings wherein like structure is indicated with like reference numerals and in which:

FIG. 1 is a block diagram depicting major components of an example system for intra-operative stimulation and neurophysiological monitoring according to aspects of the present disclosure.

FIG. 2A illustrates example anode electrode placement positions for intra-operative stimulation and neurophysiological monitoring according to aspects of the present disclosure.

FIG. 2B illustrates example cathode electrode placement positions for intra-operative stimulation and neurophysiological monitoring according to aspects of the present disclosure.

FIG. 2C illustrates example cathode and anode electrode placement positions for intra-operative stimulation and neurophysiological monitoring according to aspects of the present disclosure.

FIG. 3 illustrates an example peripheral and central stimulation pattern for intra-operative stimulation and neurophysiological monitoring according to aspects of the present disclosure.

FIG. 4 depicts aspects of an example device for intra-operative stimulation and neurophysiological monitoring according to aspects of the present disclosure.

FIG. 5 depicts a method for intra-operative stimulation and neurophysiological monitoring according to aspects of the present disclosure.

FIG. 6 depicts another method for intra-operative stimulation and neurophysiological monitoring according to aspects of the present disclosure.

### DETAILED DESCRIPTION

Aspects of the present disclosure provide apparatuses, methods, processing systems, and computer-readable mediums for intra-operative stimulation and neurophysiological monitoring.

In some aspects, the intra-operative stimulation and neurophysiological monitoring uses trans-spinal evoked potentials, posterior root muscle reflexes, root-evoked potentials, or dorsal root reflexes, in which a root sensory motor response is induced with electrical stimulation. The intra-operative stimulation and neurophysiological monitoring may use trans-spinal evoked potentials to monitor motor function in a patient's legs from key myotomes including, but not limited to, L1, L2, L3, L4, and L5 (the first, second, third, fourth, and fifth lumbar vertebrae).

The intra-operative stimulation and neurophysiological monitoring described herein may provide improvements over other techniques, such as TceMEP. The intra-operative stimulation and neurophysiological monitoring techniques described herein may be used intra-operatively, provide more frequent and better feedback for assessing the motor pathways and the feedback can be collected unilaterally, reduce false positives and false negatives, introduce less surgical disruption, are more resistant to anesthesia, and require less pulse power (e.g., 24-70 mA). Further, the intra-operative stimulation and neurophysiological monitoring described herein does not require the use of needles.

In some aspects, the intra-operative stimulation and neurophysiological monitoring uses improved positioning of the stimulation cathode electrodes. In some aspects, the intra-operative stimulation and neurophysiological monitoring uses two pairs of cathode electrodes at the T10-11 and T11-12 interspaces. As used herein, T10-11 and T11-12 are the anatomical terms referring to the vertebrae of the thoracic spine, located between the cervical spine (e.g., the neck) and the lumbar spine (e.g., the lower back). T10-11 refers to the spinal segment between the tenth and eleventh thoracic vertebrae and T11-12 refers to the spinal segment between the eleventh and twelfth thoracic vertebrae.

In some aspects, the intra-operative stimulation and neurophysiological monitoring uses improved positioning of the stimulation anode electrodes. In some aspects, the intra-operative stimulation and neurophysiological monitoring anode electrodes positioned at each anterior superior iliac crest. The anterior superior iliac crests are part of the ilium, which is one of the three bones that make up the hip bone or pelvic girdle. The iliac crests serve as attachment points for various muscles, including the abdominal muscles and the muscles of the lower back and hips. The anterior superior iliac crests are bony prominences located at the front of the pelvis and can be felt as two prominent points on either side of the abdomen, just below the waistline and slightly to the outer side.

In some aspects, the intra-operative stimulation and neurophysiological monitoring uses the intra-operative stimulation and neurophysiological monitoring uses a pair of cathode electrodes in the high thoracic or cervical spine region. The cervical spine consists of seven vertebrae numbered C1 to C7, starting from the base of the skull and extending down to the top of the thoracic spine. The cervical spine supports the head and allows for various movements of the neck. In some aspects, the pair of cathode electrodes in the high thoracic or low cervical spine region may be used in addition to the cathode electrodes in the thoracic spine region. In some aspects, the cathode electrodes are located at or between the C5 and T3. In some aspects, a pair of anode electrodes located on the back of the skull are used with the cathode electrodes in the high thoracic or low cervical spine region. For example, the pair of anode electrodes may be positioned over the temporal bones such as over the mastoid processes on each side of the patient's cranium.

In some aspects, the intra-operative stimulation and neurophysiological monitoring uses priming stimulating pulses, via peripheral electrodes positioned on lower and or upper extremities of the patient, prior to the high or low thoracic stimulation to improve the resulting muscle response signals.

In some aspects, one or more computing devices are used to automate aspects of the intra-operative stimulation and neurophysiological monitoring. In some aspect, the one or more computing devices collect the resulting muscle response signals from recording electrodes and process the resulting muscle response signals. In some aspect, the one or more computing devices optimize the central and peripheral stimulation to obtain adequate resulting muscle response signals. In some aspects, the one or more computing devices compare the resulting muscle response signals to baseline muscle response signals to detect damage or potential damage to the patient's motor pathways. In some aspects, the one or more computing devices display information associated with the neurophysiological monitoring to a display, which may include providing alerts when changes are detected.

In an illustrative embodiment, the intra-operative stimulation and neurophysiological monitoring includes, prior to starting the surgical procedure, placing the central stimulating cathode electrodes on the patient's back over the thoracic spinal regions, placing the central stimulating anode electrodes over the patient's anterior superior iliac crests, and placing the peripheral electrodes and recording electrodes on the patient's legs. The intra-operative stimulation and neurophysiological monitoring then includes establishing baseline muscle response electrical waveforms from muscles of interest for the monitoring by, for each electrode pair, ramping the stimulation intensity from an initial stimulation intensity and recording the resulting muscle response electrical waveforms from the muscles of interest. If the resulting muscle response electrical waveforms are deemed inadequate, then peripheral stimulation is performed to augment the resulting muscle response electrical waveforms. The intra-operative stimulation and neurophysiological monitoring then includes analyzing the resulting muscle response electrical waveforms to determine the optimum central (and peripheral, if used) stimulation intensity and pattern for each of the muscles of interest. The intra-operative stimulation and neurophysiological monitoring then includes intermittently performing the stimulation according to optimum stimulation intensity and pattern and monitoring for changes in the resulting muscle response electrical waveforms. The intra-operative stimulation and neurophysiological monitoring then includes triggering an alert when changes indicative of an injury to the patient's motor pathways are detected.

While aspects are described herein for intra-operative motor integrity monitoring, the techniques and apparatus described herein can also be used outside of the operating room in non-operative use cases.

### Intra-Operative Motor Integrity Monitoring System

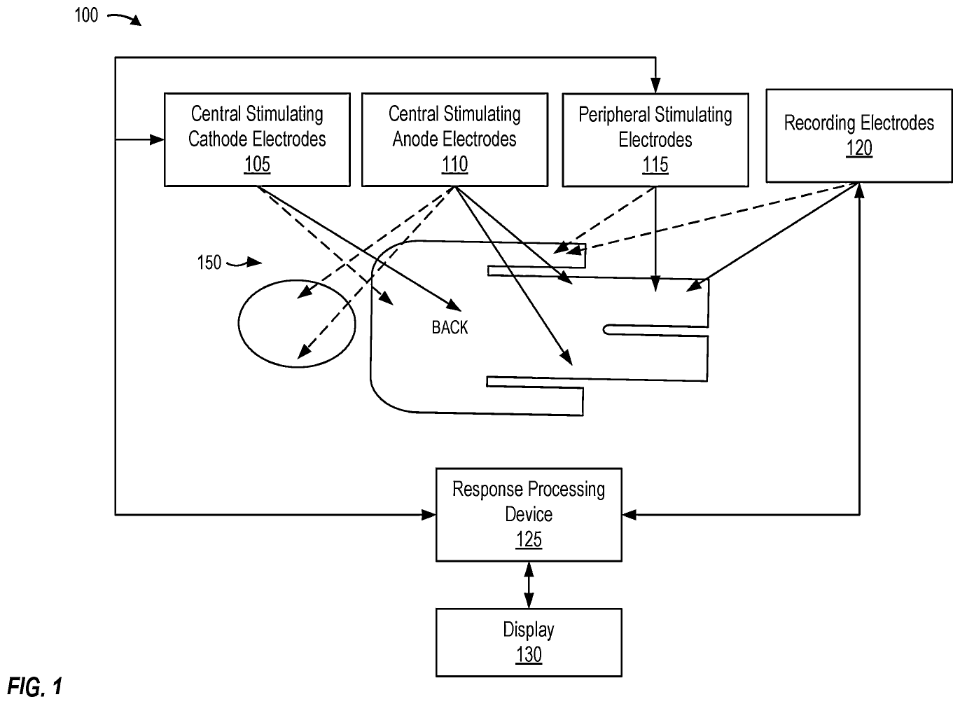

FIG. 1 is a block diagram depicting major components of an example system 100 for intra-operative stimulation and neurophysiological monitoring according to aspects of the present disclosure.

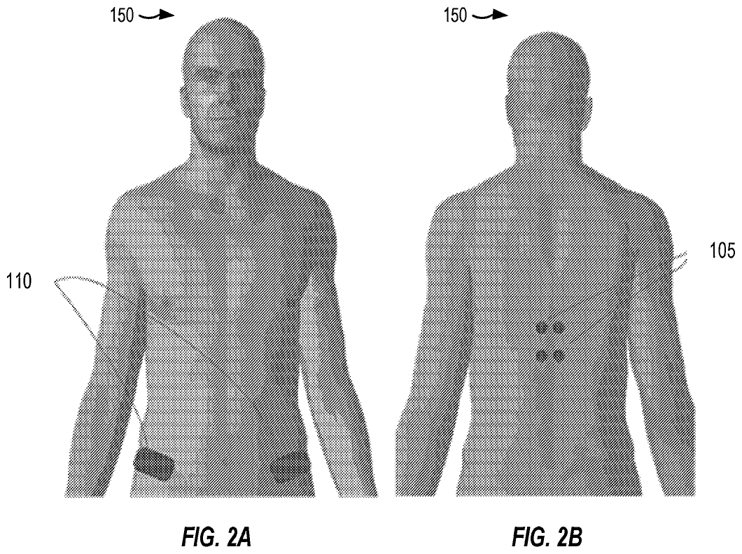

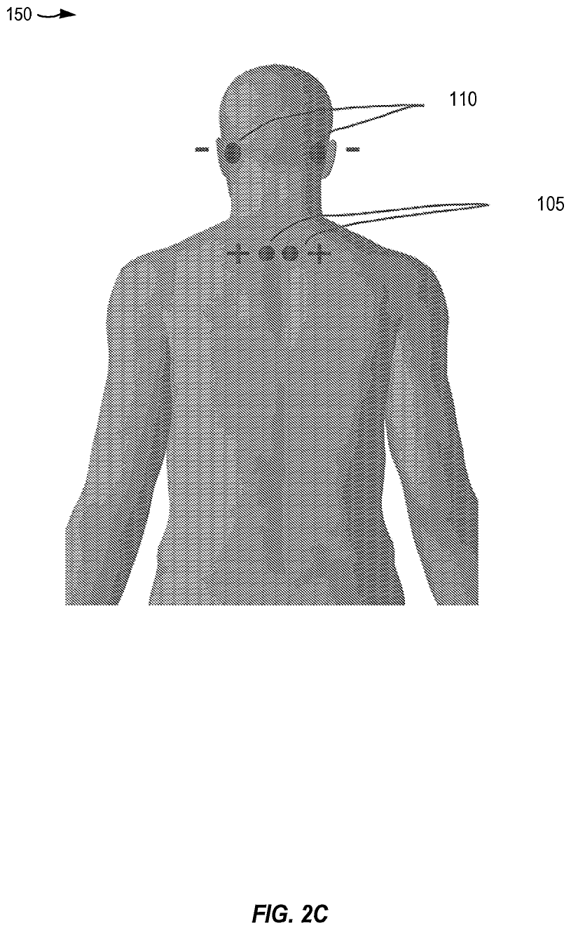

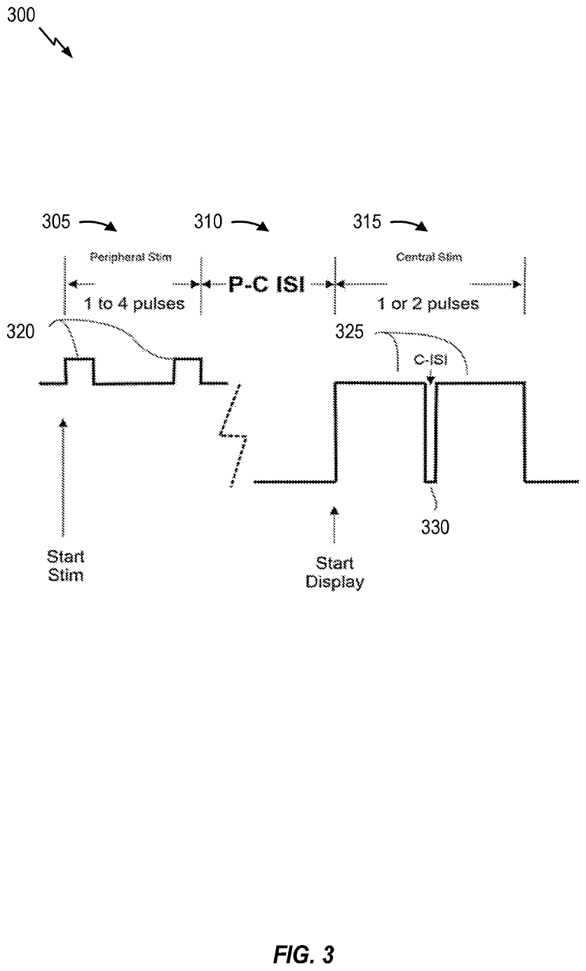

As shown in FIG. 1, the system 100 for intra-operative stimulation and neurophysiological monitoring includes one or more central stimulating cathode electrodes 105 and one or more central stimulating anode electrodes 110. The central stimulating cathode electrodes 105 and central stimulating anode electrodes 110 are described in more detail below with respect to FIGS. 2A-2C. As shown in FIG. 1, the system 100 for intra-operative stimulation and neurophysiological monitoring includes one or more peripheral stimulating electrodes 115 and one or more recording electrodes 120. Operation of the central stimulating cathode electrodes 105, central stimulating anode electrodes 110, peripheral stimulating electrodes 115, and recording electrodes 120 are described in more detail below with respect to FIG. 3. As shown in FIG. 1, the system 100 for intra-operative stimulation and neurophysiological monitoring includes a response processing device 125 and a display 130. The response processing device 125 is described in more detail below with respect to FIG. 3.

The central stimulating cathode electrodes 105 and the central stimulating anode electrodes 110 are configured to provide electrical stimulation of one or more sensory nerve roots of the patient 150 (e.g., in a supine, lateral, or prone positioning). The central stimulating cathode electrodes 105 and the central stimulating anode electrodes 110 are placed on the body of a patient 150. In some embodiments, the central stimulating cathode electrodes 105 and the central stimulating anode electrodes 110 are secured to the patient 150 cutaneously (e.g., surface electrodes). Cutaneous electrodes are non-invasive electrodes placed on the skin of the patient 150. In some embodiments, the central stimulating cathode electrodes 105 and the central stimulating anode electrodes 110 are inserted into the skin of the patient 150 subcutaneously (e.g., subdermal electrodes).

In some embodiments, a pair of, two pairs of, or more, central stimulating cathode electrodes 105 are placed on the body of the patient 150. The central stimulating cathode electrodes 105 may be placed at one or more dorsal thoracic levels near the midline. In some embodiments, the central stimulating cathode electrodes 105 may be placed at, or near, the tenth or eleventh thoracic vertebrae of the patient 150. In some embodiments, the central stimulating cathode electrodes 105 may be placed at the T10-T11 and/or the T11-T12 interspinal levels. FIG. 2B illustrates an example system 100 in which two pairs of central stimulating cathode electrodes 105 are placed at the T10-T11 and T11-T12, respectively.

The positioning of the central stimulating cathode electrodes 105 at the T10-T11 and/or the T11-T12 allows stimulation to be delivered to the back of the thorax of the patient 150.

The central stimulating anode electrodes 110 provide the conductive pathway for electrical current. For example, the central stimulating anode electrodes 110 provide the positive electrodes where the electrical current enters the body of the patient 150, while the central stimulating cathode electrodes 105 provide the negative electrodes where the electrical current exits the body of the patient 150, with the cathode(s) acting as the stimulating source.

In some embodiments, a pair of central stimulating anode electrodes 110 is used. In some embodiments, the central stimulating anode electrodes 110 are positioned at or around the anterior superior iliac crests of the patient 150. The central stimulating anode electrodes 110 may be larger in size than the central stimulating cathode electrodes 105. FIG. 2A illustrates an example system 100 in which a pair of larger surface central stimulating anode electrodes 110 are placed bilaterally over the area of the anterior superior iliac crests of the patient 150.

The larger size surface central stimulating anode electrodes 110 allows anodal dispersion of the stimulus. In some aspects, use of an electrode array, as shown in FIG. 2A, allows alternating stimulation on different sides. In addition, use of an electrode array, as shown in FIG. 2A, may provide unilateral or serial bilateral information with low power stimulation and provides a stable waveform.

FIG. 2C illustrates another example placement of the central stimulating cathode electrodes 105 and central stimulating anode electrodes 110. As shown in FIG. 2C, a pair of central stimulating cathode electrodes 105 may be located at the high thoracic or low cervical spine region of the patient 150. For example, the central stimulating cathode electrodes 105 may be located at or between any of the C5 to T3 vertebrae of the patient 150. In some embodiments, the stimulating electrodes are preferably placed at the T1-T2 interspace. As shown in FIG. 2C, a pair of central stimulating anode electrodes 110 may be positioned at the back of the skull of the patient 150 for use with the central stimulating cathode electrodes 105 in the cervical spine region of the patient 150. For example, the pair of anode electrodes may be positioned over the temporal bones on each side of the cranium of the patient 150. In some embodiments, these electrodes are preferably placed over the mastoid process.

In some aspects, the pair of central stimulating cathode electrodes 105 in the high thoracic or low cervical spine region and pair of the central stimulating anode electrodes 110 on the back of the skull of the patient 150, shown in FIG. 2C, may be used alone or in addition to the central stimulating cathode electrodes 105 over the thoracic spine region and the central stimulating anode electrodes 110 over the anterior superior iliac crests of the patient 150, shown in FIGS. 2A-2B.

The electrical stimulations provided by the central stimulating cathode electrodes 105 and the central stimulating anode electrodes 110 may cause one or more muscles of the body of the patient 150 to provide responses. The muscle responses may be detected as resultant electrical waveforms from depolarized supplying motor nerves of those muscles. The muscle responses may be reflexive, direct, or both. The resultant electrical waveforms from the stimulated muscles are collected by recording electrodes 120.

In some embodiments, the recording electrodes 120 are attached, connected, and/or coupled over one or more muscles of the upper or lower extremities of the patient 150. For example, the recording electrodes 120 may be positioned at one or more leg muscles of either or both legs of the patient 150 and/or the recording electrodes 120 may be positioned at one or more upper extremities of the patient 150, such as on both arms of the patient 150, such as on the wrists or near the ulnar nerve. The recording electrodes 120 are in communication with the response processing device 125.

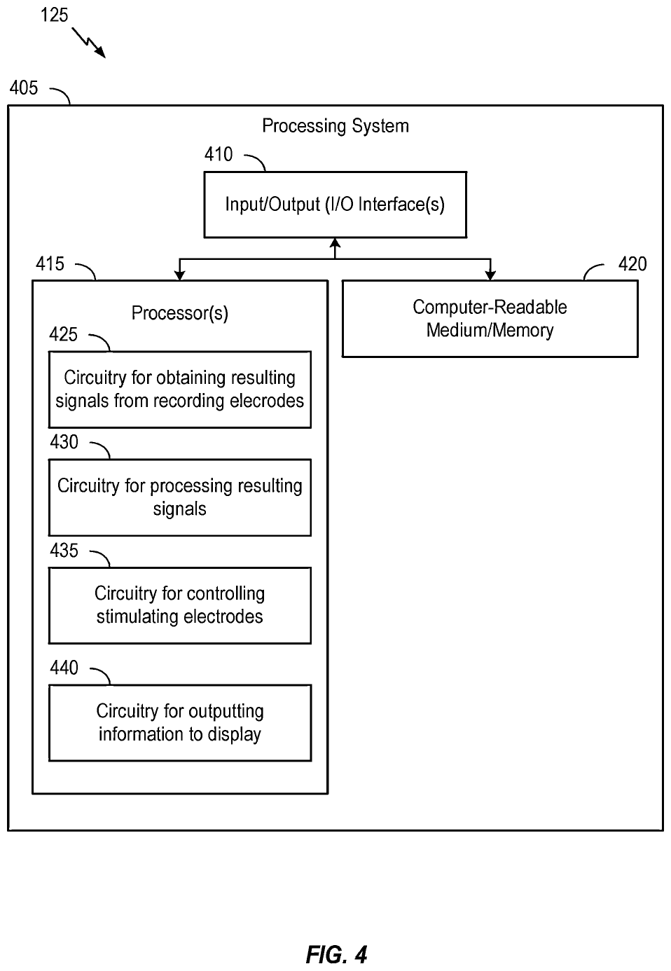

In some embodiments, the resulting response(s) from the stimulated muscles is improved through the use one or more priming pulses via the peripheral stimulating electrodes 115. In some embodiments, the peripheral stimulating electrodes 115 are located on one or more of the upper or lower extremities of the patient 150. For example, a priming pulse may be provided to a peripheral nerve in one or both of the legs (or arms) of the patient 150. The priming pulse is an electrical stimulation delivered, by the peripheral stimulating electrodes 115, prior to the central electrode stimulation (e.g., 60-120 ms prior to the central electrode stimulation). According to certain aspects, the priming pulse may be applied when a preceding central stimulation did not provide an adequate muscle response signaling. As described in more detail below with respect to the FIG. 4, aspects of the central and peripheral priming stimulation may be controlled automatically by the response processing device 125.

FIG. 3 illustrates an example peripheral and central stimulation pattern 300 for intra-operative stimulation and neurophysiological monitoring according to aspects of the present disclosure.

As shown in FIG. 3, the peripheral stimulation, via peripheral stimulating electrodes 115, may be performed during a duration 305 (e.g., around 300 μs). The peripheral stimulation may include a number of stimulating priming electrical pulses (e.g., 1-4 pulses). The number of priming pulses, the duration and intensity of the priming pulses, and the frequency of the priming pulses may be automatically calculated (e.g., by response processing device 125, as discussed in more detail below with respect to FIG. 4).

The peripheral stimulating may precede the central stimulation by an interval 310 referred herein as the peripheral-central inter-stimulus interval (P-C ISI). The P-C ISI 310 may have a duration of between 50 ms to 140 ms.

After the P-C ISI 310, the central thoracic stimulation, via central stimulating cathode electrodes 105 and central stimulating anode electrodes 110, may be performed during a duration 315. The central stimulation may include one or two electrical pulses. In some embodiments, the central stimulation delivers at least one pulse having a duration of at least 500 μs. A central inter-stimulus interval (C-ISI) is defined between central stimulation pulses. In some embodiments, the central stimulation pulses are delivered to one side of the patient 150. In some embodiments, the central stimulation pulses are delivered to both sides of the patient 150 sequentially.

The number of priming and central stimulation pulses, the duration and intensity of the priming and central stimulation pulses, the frequency of the priming and central stimulation pulses, whether to use peripheral stimulation, and adequacy of resulting muscle responses may be automatically determined (e.g., calculated or selected) by response processing device 125, as discussed in more detail below with respect to FIG. 4.

FIG. 4 depicts aspects of an example response processing device 125 for intra-operative stimulation and neurophysiological monitoring according to aspects of the present disclosure. As shown, the response processing device 125 includes a processing system 405 with a number of input/output interfaces 410, processors 415, and computer-readable memory 420. While shown as a single device, in some embodiments, functions of response processing device 125 may be distributed across multiple physical devices.

I/O interfaces 410 may connect the response processing device 125, wired or wirelessly, to recording electrodes 120 to enable response processing device 125 to obtain resulting muscle response electrical waveforms from recording electrodes 120 and/or to provide control to recording electrodes 120.

I/O interfaces 410 may connect the response processing device 125, wired or wirelessly, to central stimulating cathode electrodes 105, central stimulating anode electrodes 110, and/or peripheral stimulating electrodes 115 to enable response processing device 125 to control the central stimulating cathode electrodes 105, central stimulating anode electrodes 110, and/or peripheral stimulating electrodes 115.

I/O interfaces 410 may connect the response processing device 125, wired or wirelessly, to display 130 to enable response processing device 125 to display neurophysiological monitoring information generated based on processing the resulting muscle response electrical waveforms to the stimulation.

I/O interfaces 410 may provide one or more user interfaces enabling a user to interact with the response processing device 125, such as buttons, touch screens, switches, keyboards, mouse, or other user interfaces.

As shown in FIG. 4, the processing system 405 of response processing device 125 includes one or more processors 415 and computer-readable media/memory 420. The processor(s) 415 may include circuitry for performing functions of the response processing device 125. The computer-readable media/memory 420 may include instructions, executable by the one or more processor(s) 415 to perform the functions of the response processing device 125. The computer-readable media/memory 420 may store other information, such as collected data (e.g., resulting muscle response electrical waveform data) and information generated from the data.

In some embodiments, the processor(s) include circuitry 425 for obtaining resulting signals from recording electrodes 120.

In some embodiments, the processor(s) include circuitry 430 for processing the resulting signals.

In some aspects, processing the resulting signals includes averaging the muscle response electrical waveforms to several stimuli together to reduce noise, improve the signal-to-noise ratio, and produce a clean signal.

In some aspects, the response processing device 125 processing the resulting signals includes automatically determining adequacy of the resulting signals. In some aspects, the response processing device 125 determines adequacy of both anterior and posterior root activation responses. The response processing device 125 may compare the anterior root activation responses to the posterior root activation responses. The anterior root activation responses provide activation responses of the axons as they exit the spinal cord and travel to the muscle. Comparing the anterior and posterior root activation response can help identify where an injury or potential injury has occurred, and how significant the injury or potential injury is.

The response processing device 125 determines adequacy of the resulting signals based on a calculation of one or more parameters and comparing the parameters to one or more configured thresholds. The response processing device 125 may calculate latency of onset peak of the resulting waveforms, behavior of the resulting waveforms over increased central stimulation or suppression of the resulting waveforms (e.g., by paired stimulation pulses delivered 30-40 ms apart).

In some embodiments, the processor(s) include circuitry 435 for controlling the stimulating electrodes based on the processing of the resulting signals.

In some aspects, the response processing device 125 controls the central stimulation by optimizing the central stimulation pulses. The response processing device 125 may optimize the central stimulation intensity that elicits reflexive responses over anterior root responses by adjusting the latency, amplitude, and response of the stimulating pulses. For example, the response processing device 125 may automatically gradually increase (e.g., ramping) the central stimulation until an adequate muscle response is identified by the response processing device 125 or a maximal stimulation is reached or an adequate comparison of anterior root and posterior root responses is present. Increasing the central stimulation may include increasing the intensity, number, duration, and/or frequency of the stimulation pulses delivered by central stimulating cathode electrodes 105 and central stimulating anode electrodes 110 during the central stimulation duration 315.

In some aspects, the response processing device 125 controls the central stimulation by automatically alternating between sides (e.g., which central stimulating anode electrode 110 is used) or performing stimulation using only one of the central stimulating anode electrodes 110 independently.

In some aspects, where multiple recording electrodes 120 record resulting signals from multiple different muscles of the patient 150, the response processing device 125 may optimize the central stimulation separately for each muscle being recorded. For example, larger fibers tend to have lower rheobase (the minimum stimulation intensity with prolonged pulse duration which just reaches threshold activation of excitable tissue, such as muscles or nerves) values than smaller fibers. Thus, a longer stimulation pulse duration with lower pulse intensity may be used for larger fibers while avoiding stimulation of the smaller fibers. Further, tissue geometry and relative conductance can vary for different activation of different muscles and, therefore, different stimulation may be used. The stimulation may also be varied based on proximity to the nerve.

In some aspects, the response processing device 125 controls the central thoracic stimulation by automatically determining the optimal spinal level to stimulate where multiple central stimulating cathode electrodes 105 are coupled to the patient 150.

In some aspects, the response processing device 125 controls the peripheral stimulation. For example, the response processing device 125 may automatically determine whether to apply a priming pulse based on whether a prior central stimulation results in adequate anterior and/or posterior muscle activation responses. In some aspects, the response processing device 125 may iteratively adapt the peripheral stimulation (e.g., adapt the number, duration, intensity, and/or frequency of the priming pulses) until adequate anterior and/or posterior root muscle activation responses are obtained or a maximal peripheral stimulation is reached.

In some embodiments, the processor(s) include circuitry 440 for outputting information, generated based on processing the resulting signals, to the display 130.

In some aspects, the display 130 displays the resulting muscle response electrical waveforms. In some aspects, the display 130 displays an anatomical diagram depicting the location of a detected change in the resulting muscle response electrical waveforms to the display 130.

In some aspects, the response processing device 125 automatically detects and identifies impending neural injury based on changes in the resulting muscle response electrical waveform signals. For example, the response processing device 125 may automatically detect and identify a lumbar motor nerve injury during a lumbar surgery.

The response processing device 125 may identify changes in neural function by comparing baseline muscle responses (e.g., based on resultant electrical waveforms obtained pre-surgery or at the beginning of the surgical procedure) to those obtained during surgery. For example, the response processing device 125 may compare the amplitude, latency, morphology, and/or area under the curve of the baseline and intra-operative muscle response electrical waveforms.

The response processing device 125 may further identify changes in neural function by taking into account information from an anesthesia machine or a neuromuscular testing machine. Based on the comparison, the response processing device 125 can determine whether changes in the resulting electrical waveforms are due to anesthesia or the presence of paralytic agents.

The response processing device 125 may further identify changes in neural function by taking into account information from a blood pressure machine and/or a pulse oximetry machine to determine whether changes in the resulting muscle response electrical waveforms are due to blood pressure or perfusion.

In some aspects, the response processing device 125 compares resulting muscle response electrical waveforms from different limbs of the patient 150.

In some aspects, the response processing device 125 outputs an alert to a use when changes are identified in the resulting muscle response electrical waveforms. For example, the alert may output to the display 130, output directly from the response processing devices, or output to another device. The alert may be a visual alert, a text alert, an audible sound or message, an alarm, a light, a color coding, or other type of alert.

In some aspects, the response processing device 125 generates and outputs a recommendation for action to ameliorate the identified neural dysfunction.

In some aspects, response processing device 125 accounts for changes in the resulting muscle response electrical waveforms that are due to systemic effects of paralytic use, anesthesia, or blood pressure changes to eliminate alerts that are false positives.

In some aspects, the display 130 displays information, such as, but not limited to, the body areas of the patient 150 being stimulated and recorded from, baseline and current signal traces, trends in signals, relevant changes in signals, location of signal changes, quality of recorded signals, position of electrodes, and alerts due to significant changes in signal. The display 130 may include, but not is limited to, multiple buttons or control inputs. The buttons or inputs may allow an operator to set up the initial monitoring layout and to interact with the display 130 during monitoring to add additional information or respond to alerts. In some aspects, display 130 may allow override of a change in signal by an anesthesiologist, or other medical personnel, such as when a signal change is related to an event unrelated to nerve injury.

In some embodiments, the response processing device 125 receives a user input regarding the accuracy of the resulting muscle response electrical waveforms.

In some aspects, the response processing device 125 includes one or more other components (not shown), such as electric stimulators, pre-amplifiers, amplifiers, and/or computer components.

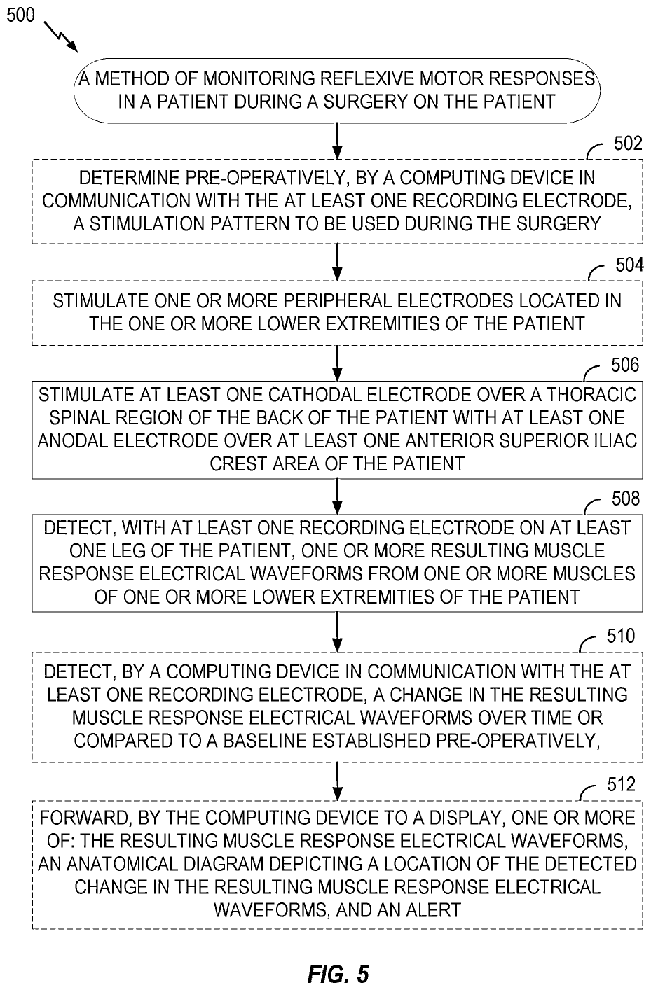

FIG. 5 depicts a method 500 of monitoring reflexive motor responses in a patient (e.g., patient 150) during a surgery (e.g., a lumbar surgery) on the patient according to aspects of the present disclosure. In one aspect, the patient is in a supine position.

Optionally, the method 500 begins at operation 502 with determining pre-incision operatively, by a computing device (e.g., response processing device 125), a first stimulation pattern to be used during the surgery. In one aspect, method 500 optionally includes determining, by the computing device during the surgery, a second stimulation pattern to be used during the surgery.

In one aspect, the method 500 optionally includes determining, by the computing device, one or more of: a number of pulses, a duration of the pulses, a frequency of the pulses, and an intensity of the pulses used to stimulate the at least one cathodal electrode with the at least one anodal electrode at operation 506. In one aspect, the determining is based on one or more of: a proximity, a tissue geometry, and a conductance of current and the one or more muscles to be monitored. In one aspect, the determining is based on a target latency and amplitude of the one or more resulting muscle response electrical waveforms in response to the stimulating. In one aspect, the determining includes using a first intensity of the pulses used to stimulate the at least one cathodal electrode with the at least one anodal electrode; when a first latency and amplitude of a first one or more resulting muscle response electrical waveforms does not satisfy the target latency and amplitude, using a second intensity of the pulses used to stimulate the at least one cathodal electrode with the at least one anodal electrode, wherein the second intensity is higher than the first intensity; and when the first latency and amplitude of the first one or more resulting muscle response electrical waveforms satisfies the target latency and amplitude, using the first intensity of the pulses used to stimulate the at least one cathodal electrode with the at least one anodal electrode.

In one aspect, the method 500 optionally includes receiving input from a user to perform stimulating the at least one cathodal electrode with the at least one anodal electrode at operation 506, wherein the stimulating at operation 506 is in response to the user input.

In one aspect, method 500 optionally includes, at operation 504, stimulating one or more peripheral electrodes (e.g., peripheral stimulating electrodes 115) located in the one or more upper or lower extremities of the patient prior to stimulating the at least one cathodal electrode (e.g., central stimulating cathode electrodes 105) with the at least one anodal electrode (e.g., central stimulating anode electrodes 110) at operation 506. In one aspect, method 500 optionally includes selecting the one or more peripheral electrodes to stimulate to enhance the one or more resulting muscle response electrical waveforms from the one or more muscles of the one or more lower extremities of the patient.

In one aspect, method 500 optionally includes determining, by the computing device, to stimulate the one or more peripheral electrodes at operation 504 based on determining inadequacy of one or more previous resulting muscle response electrical waveforms from the one or more muscles of the one or more lower extremities of the patient. In one aspect, method 500 optionally includes determining, by the computing device, one or more of: a number of priming pulses, a duration of the priming pulses, a frequency of the priming pulses, and an intensity of the priming pulses used to stimulate the one or more peripheral electrodes at operation 504.

Method 500 includes, at operation 506, stimulating at least one cathodal electrode over a thoracic spinal region of the back (e.g., T10, T11, T12) of the patient with at least one anodal electrode over at least one anterior superior iliac crest area of the patient. In one aspect, at least one of a pair of cathodal electrodes over a high thoracic or cervical spinal region of the back of the patient are stimulated with at least one anodal electrode of a pair of anodal electrodes on the patient's skull (e.g., over each temporal bone).

In one aspect, stimulating the at least one cathodal electrode at operation 506 includes stimulating the at least one cathodal electrode cutaneously. In one aspect, stimulating the at least one cathodal electrode at operation 506 includes stimulating the at least one cathodal electrode subcutaneously.

In one aspect, the at least one cathodal electrode is located at or between the tenth, eleventh, or twelfth thoracic vertebrae of the patient.

In one aspect, the at least one cathodal electrode comprises a first pair of cathodal electrodes located on opposite sides of the thoracic spinal region of the back of the patient. In one aspect, the at least one cathodal electrode further comprises a second pair of cathodal electrodes located on opposite sides of the thoracic spinal region of the back of the patient above (i.e., cranially) or below (i.e., caudally) the first pair of cathodal electrodes. In one aspect, stimulating the at least one cathodal electrode at operation 506 includes alternating between stimulating the first pair of cathodal electrodes and the second pair of cathodal electrodes. In one aspect, method 500 optionally includes determining, by a computing device in communication with the at least one recording electrode, the at least one cathodal electrode to stimulate, from a plurality of cathodal electrodes over the thoracic spinal region of the back of the patient. In one aspect, determining the at least one cathodal electrode to stimulate includes determining the at least one cathodal electrode to stimulate based on the one or more muscles to be monitored.

In one aspect, stimulating the at least one cathodal electrode at operation 506 includes stimulating the at least one cathodal electrode with the at least one anodal electrode using one or more electrical pulses having a pulse duration of 500 μs or longer.

In one aspect, the stimulating the at least one cathodal electrode at operation 506 is performed after an inter-stimulus interval (e.g., P-C ISI 310) after the stimulating the one or more peripheral electrodes at operation 504.

In one aspect, the at least one anodal electrode comprises a pair of anodal electrodes positioned over both anterior superior iliac crest areas of the patient. In one aspect, stimulating the at least one cathodal electrode at operation 506 includes alternating stimulating with a first anodal electrode of the pair of anodal electrodes positioned over a first anterior superior iliac crest area of the patient and a second anodal electrode of the pair of anodal electrodes positioned over a second anterior superior iliac crest area of the patient.

In one aspect, the at least one recording electrode is coupled to at least one muscle of the one or more lower extremities of the patient.

Method 500 proceeds to, at operation 508, detecting, with at least one recording electrode (e.g., recording electrodes 120) on at least one leg of the patient, one or more resulting muscle response electrical waveforms from one or more muscles of one or more lower extremities of the patient.

In one aspect, method 500 optionally includes, at operation 510, detecting, by the computing device, a change in the resulting muscle response electrical waveforms over time or compared to a baseline established pre-operatively, wherein the change comprises one or more of: a changed latency, amplitude, and morphology of the resulting muscle response electrical waveforms.

In one aspect, method 500 optionally includes identifying, by the computing device, an injury or potential injury along a motor pathway associated with the one or more muscles.

In one aspect, method 500 optionally includes, at operation 512, forwarding, by the computing device to a display (e.g., display 130), one or more of: the resulting muscle response electrical waveforms, an anatomical diagram depicting a location of the detected change in the resulting muscle response electrical waveforms, and an alert associated with the identified injury or potential injury.

In one aspect, method 500 optionally includes generating a recommended action to ameliorate the identified injury or potential injury.

In one aspect, method 500 optionally includes obtaining information from one or more of: an anesthesia machine, a neuromuscular junction testing machine, an oximetry machine, and a blood pressure machine; and determining, based on the information, whether the changes in the resulting muscle response electrical waveforms are due to one or more of: anesthesia, a paralytic effect, blood pressure, and perfusion changes.

In one aspect, method 500 optionally includes averaging multiple resulting muscle response electrical waveforms obtained in response to performing multiple stimulations of the at least one cathodal electrode with the at least one anodal electrode to reduce noise.

In one aspect, method 500 optionally includes receiving a user input indicating an accuracy of the resulting muscle response electrical waveforms.

FIG. 5 is just one example of a method, and other methods including fewer, additional, or alternative steps are possible consistent with this disclosure.

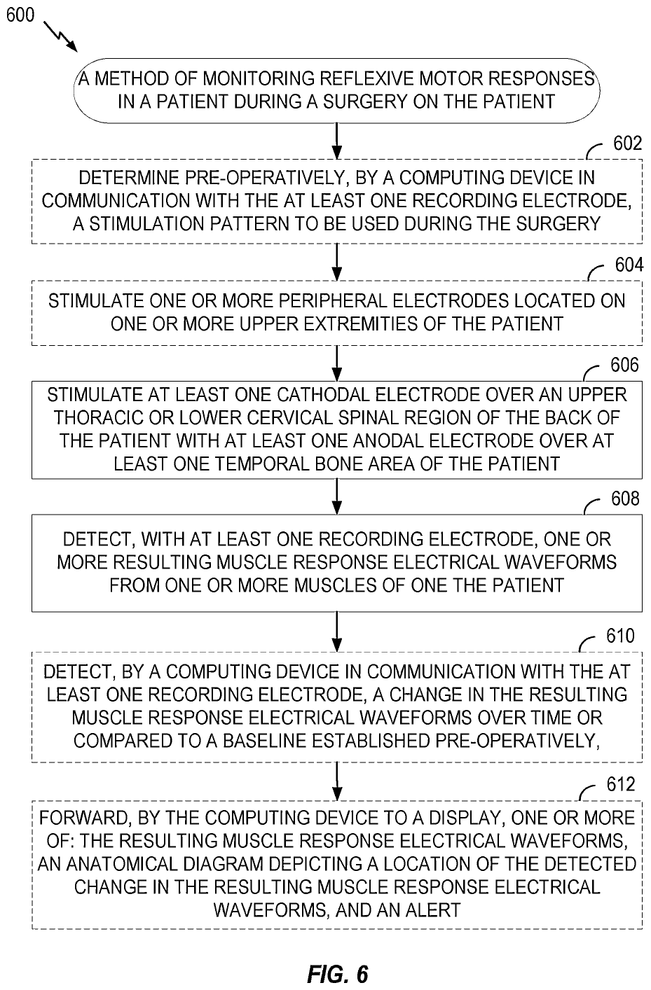

FIG. 6 depicts a method 600 of monitoring reflexive motor responses in a patient (e.g., patient 150) during a surgery (e.g., a lumbar surgery) on the patient according to aspects of the present disclosure. In one aspect, the patient is in a supine position.

Optionally, the method 600 begins at operation 602 with determining pre-operatively, by a computing device (e.g., response processing device 125), a first stimulation pattern to be used during the surgery. In one aspect, method 600 optionally includes determining, by the computing device during the surgery, a second stimulation pattern to be used during the surgery.

In one aspect, the method 600 optionally includes determining, by the computing device, one or more of: a number of pulses, a duration of the pulses, a frequency of the pulses, and an intensity of the pulses used to stimulate the at least one cathodal electrode with the at least one anodal electrode at operation 606. In one aspect, the determining is based on one or more of: a proximity, a tissue geometry, and a conductance of current and the one or more muscles to be monitored. In one aspect, the determining is based on a target latency and amplitude of the one or more resulting muscle response electrical waveforms in response to the stimulating. In one aspect, the determining includes using a first intensity of the pulses used to stimulate the at least one cathodal electrode with the at least one anodal electrode; when a first latency and amplitude of a first one or more resulting muscle response electrical waveforms does not satisfy the target latency and amplitude, using a second intensity of the pulses used to stimulate the at least one cathodal electrode with the at least one anodal electrode, wherein the second intensity is higher than the first intensity; and when the first latency and amplitude of the first one or more resulting muscle response electrical waveforms satisfies the target latency and amplitude, using the first intensity of the pulses used to stimulate the at least one cathodal electrode with the at least one anodal electrode.

In one aspect, the method 600 optionally includes receiving input from a user to perform stimulating the at least one cathodal electrode with the at least one anodal electrode at operation 606, wherein the stimulating at operation 606 is in response to the user input.

In one aspect, method 600 optionally includes, at operation 604, stimulating one or more peripheral electrodes (e.g., peripheral stimulating electrodes 115) located in the one or more extremities of the patient (e.g., such as one the arms, wrist, or hands) prior to stimulating the at least one cathodal electrode (e.g., central stimulating cathode electrodes 105) with the at least one anodal electrode (e.g., central stimulating anode electrodes 110) at operation 606. In one aspect, method 600 optionally includes selecting the one or more peripheral electrodes to stimulate to enhance the one or more resulting muscle response electrical waveforms from the one or more muscles of the one or more extremities of the patient.

In one aspect, method 600 optionally includes determining, by the computing device, to stimulate the one or more peripheral electrodes at operation 604 based on determining inadequacy of one or more previous resulting muscle response electrical waveforms from the one or more muscles of the one or more extremities of the patient. In one aspect, method 600 optionally includes determining, by the computing device, one or more of: a number of priming pulses, a duration of the priming pulses, a frequency of the priming pulses, and an intensity of the priming pulses used to stimulate the one or more peripheral electrodes at operation 604.

Method 600 includes, at operation 606, stimulating at least one cathodal electrode over a high thoracic or low cervical spinal region of the back of the patient with at least one anodal electrode over at least one temporal bone of the patient. In one aspect, at least one of a pair of cathodal electrodes over a high thoracic or low cervical spinal region of the back of the patient are stimulated with at least one anodal electrode of a pair of anodal electrodes on the patient's skull (e.g., over each temporal bone).

In one aspect, stimulating the at least one cathodal electrode at operation 506 includes stimulating the at least one cathodal electrode cutaneously. In one aspect, stimulating the at least one cathodal electrode at operation 606 includes stimulating the at least one cathodal electrode subcutaneously.

In one aspect, the method includes additional stimulating at least one cathodal electrode located at or between the tenth, eleventh, or twelfth thoracic vertebrae of the patient. In one aspect, the at least one cathodal electrode comprises a first pair of cathodal electrodes located on opposite sides of the thoracic spinal region of the back of the patient. In one aspect, the at least one cathodal electrode further comprises a second pair of cathodal electrodes located on opposite sides of the thoracic spinal region of the back of the patient above (i.e., cranially) or below (i.e., caudally) the first pair of cathodal electrodes.

In one aspect, stimulating the at least one cathodal electrode at operation 606 includes alternating between stimulating the first pair of cathodal electrodes and the second pair of cathodal electrodes. In one aspect, method 600 optionally includes determining, by a computing device in communication with the at least one recording electrode, the at least one cathodal electrode to stimulate, from a plurality of cathodal electrodes over the high thoracic or low cervical spinal region of the back of the patient. In one aspect, determining the at least one cathodal electrode to stimulate includes determining the at least one cathodal electrode to stimulate based on the one or more muscles to be monitored.

In one aspect, stimulating the at least one cathodal electrode at operation 606 includes stimulating the at least one cathodal electrode with the at least one anodal electrode using one or more electrical pulses having a pulse duration of 500 μs or longer.

In one aspect, the stimulating the at least one cathodal electrode at operation 606 is performed after an inter-stimulus interval (e.g., P-C ISI 310) after the stimulating the one or more peripheral electrodes at operation 604.

In one aspect, the at least one anodal electrode comprises a pair of anodal electrodes positioned over both temporal bones of the patient. In one aspect, stimulating the at least one cathodal electrode at operation 606 includes alternating stimulating with a first anodal electrode of the pair of anodal electrodes positioned over a first temporal bone of the patient and a second anodal electrode of the pair of anodal electrodes positioned over a second temporal bone of the patient.

In one aspect, the at least one recording electrode is coupled to at least one muscle of the one or more lower extremities of the patient.

Method 600 proceeds to, at operation 608, detecting, with at least one recording electrode (e.g., recording electrodes 120) on the patient, one or more resulting muscle response electrical waveforms from one or more muscles of one or more extremities of the patient.

In one aspect, method 600 optionally includes, at operation 610, detecting, by the computing device, a change in the resulting muscle response electrical waveforms over time or compared to a baseline established pre-operatively, wherein the change comprises one or more of: a changed latency, amplitude, and morphology of the resulting muscle response electrical waveforms.

In one aspect, method 600 optionally includes identifying, by the computing device, an injury or potential injury along a motor pathway associated with the one or more muscles.

In one aspect, method 600 optionally includes, at operation 612, forwarding, by the computing device to a display (e.g., display 130), one or more of: the resulting muscle response electrical waveforms, an anatomical diagram depicting a location of the detected change in the resulting muscle response electrical waveforms, and an alert associated with the identified injury or potential injury.

In one aspect, method 600 optionally includes generating a recommended action to ameliorate the identified injury or potential injury.

In one aspect, method 600 optionally includes obtaining information from one or more of: an anesthesia machine, a neuromuscular junction testing machine, an oximetry machine, and a blood pressure machine; and determining, based on the information, whether the changes in the resulting muscle response electrical waveforms are due to one or more of: anesthesia, a paralytic effect, blood pressure, and perfusion changes.

In one aspect, method 600 optionally includes averaging multiple resulting muscle response electrical waveforms obtained in response to performing multiple stimulations of the at least one cathodal electrode with the at least one anodal electrode to reduce noise.

In one aspect, method 600 optionally includes receiving a user input indicating an accuracy of the resulting muscle response electrical waveforms.

### EMBODIMENTS

Embodiment 1: A method of monitoring reflexive motor responses in a patient during a surgery on the patient, the method comprising: stimulating with one or more pulses a thoracic spinal region of a back of the patient using at least one cathodal electrode positioned thereon and at least one anodal electrode positioned over at least one anterior superior iliac crest area of the patient, where the one or more pulses has a duration of at least 500 μs; and detecting, with at least one recording electrode on at least one leg of the patient, one or more resulting muscle response electrical waveforms from one or more muscles of one or more lower extremities of the patient.

Embodiment 2: The method of embodiment 1, wherein stimulating the at least one cathodal electrode comprises stimulating the at least one cathodal electrode cutaneously.

Embodiment 3: The method of any combination of embodiments 1-2, wherein stimulating the at least one cathodal electrode comprises stimulating the at least one cathodal electrode subcutaneously.

Embodiment 4: The method of any combination of embodiments 1-3, wherein the at least one cathodal electrode is located at or between the tenth, eleventh, or twelfth thoracic vertebrae of the patient.

Embodiment 5: The method of any combination of embodiments 1-4, further comprising a pair of cathodal electrodes located at the high thoracic or low cervical spinal region of the patient.

Embodiment 6: The method of any combination of embodiments 1-5, wherein the at least one cathodal electrode comprises a first pair of cathodal electrodes located on opposite sides of the thoracic spinal region of the back of the patient.

Embodiment 7: The method of embodiment 6, wherein the at least one cathodal electrode further comprises a second pair of cathodal electrodes located on opposite sides of the thoracic spinal region of the back of the patient above or below the first pair of cathodal electrodes.

Embodiment 8: The method of embodiment 7, wherein the stimulating the at least one cathodal electrode comprises alternating between stimulating the first pair of cathodal electrodes and the second pair of cathodal electrodes.

Embodiment 9: The method of any combination of embodiments 1-8, further comprising: determining, by a computing device in communication with the at least one recording electrode, the at least one cathodal electrode to stimulate, from a plurality of cathodal electrodes over the thoracic spinal region of the back of the patient.

Embodiment 10: The method of embodiment 9, wherein determining the at least one cathodal electrode to stimulate comprises determining the at least one cathodal electrode to stimulate based on the one or more muscles to be monitored.

Embodiment 11: The method of any combination of embodiments 1-10, further comprising a pair of anodal electrodes positioned over the cranium of the patient.

Embodiment 12: The method of any combination of embodiments 1-11, wherein the pair of anodal electrodes are positioned over both temporal bones of the cranium of the patient.

Embodiment 13: The method of any combination of embodiments 1-12, wherein the at least one anodal electrode comprises a pair of anodal electrodes positioned over both anterior superior iliac crest areas of the patient.

Embodiment 14: The method of any combination of embodiments 1-13, wherein stimulating at least one cathodal electrode with the at least one anodal electrode comprises alternating stimulating with a first anodal electrode of a pair of anodal electrodes and a second anodal electrode of the pair of anodal electrodes.

Embodiment 15: The method of any combination of embodiments 1-14, wherein the at least one recording electrode is coupled to at least one muscle of the one or more lower extremities of the patient.

Embodiment 16: The method of any combination of embodiments 1-15, further comprising: prior to the stimulating the at least one cathodal electrode with the at least one anodal electrode, stimulating one or more lower extremities of the patient using one or more peripheral electrodes located on the one or more lower extremities.

Embodiment 17: The method of embodiment 16, further comprising selecting the one or more peripheral electrodes to stimulate to enhance the one or more resulting muscle response electrical waveforms from the one or more muscles of the one or more lower extremities of the patient.

Embodiment 18: The method of any combination of embodiments 16-17, wherein the stimulating using the at least one cathodal electrode is performed after an inter-stimulus interval (ISI) after the stimulating using the one or more peripheral electrodes.

Embodiment 19: The method of any combination of embodiments 1-18, further comprising: determining, by a computing device in communication with the at least one recording electrode, an inadequacy of one or more resulting muscle response electrical waveforms from the one or more muscles of the one or more lower extremities of the patient; and stimulating the one or more lower extremities of the patient using one or more peripheral electrodes in response to the determination.

Embodiment 20: The method of any combination of embodiments 16-19, further comprising determining, by the computing device, one or more of: a number of priming pulses, a duration of the priming pulses, a frequency of the priming pulses, and an intensity of the priming pulses used to stimulate the one or more peripheral electrodes.

Embodiment 21: The method of any combination of embodiments 1-20, further comprising determining, by a computing device in communication with the at least one recording electrode, one or more of: a number of pulses, a duration of the pulses, a frequency of the pulses, and an intensity of the pulses used to stimulate using the at least one cathodal electrode with the at least one anodal electrode.

Embodiment 22: The method of embodiment 21, wherein the determining is based on a target latency and amplitude of one or more components of the resulting muscle response electrical waveforms in response to the stimulating.

Embodiment 23: The method of embodiment 22, wherein the determining comprises: using a first intensity of the pulses used to stimulate using the at least one cathodal electrode with the at least one anodal electrode; when a first latency and amplitude of a first one or more resulting muscle response electrical waveforms does not satisfy the target latency and amplitude, using a second intensity of the pulses used to stimulate the at least one cathodal electrode with the at least one anodal electrode, wherein the second intensity is higher than the first intensity; and when the first latency and amplitude of the first one or more resulting components of the muscle response electrical waveforms satisfies the target latency and amplitude, using the first intensity of the pulses used to stimulate the at least one cathodal electrode with the at least one anodal electrode.

Embodiment 24: The method of any combination of embodiments 1-23, further comprising: determining pre-operatively, by a computing device in communication with the at least one recording electrode, a first stimulation pattern to be used during the surgery; and determining, during the surgery, a second stimulation pattern to be used during the surgery.

Embodiment 25: The method of any combination of embodiments 1-24, further comprising receiving input from a user to perform the stimulating the at least one cathodal electrode with the at least one anodal electrode, wherein the stimulating is in response to the user input.

Embodiment 26: The method of any combination of embodiments 1-25, further comprising detecting, by a computing device in communication with the at least one recording electrode, a change in the resulting muscle response electrical waveforms and their components over time or compared to a baseline established pre-operatively, wherein the change comprises one or more of: a changed latency, amplitude, and morphology of the resulting muscle response electrical waveforms and their components.

Embodiment 27: The method of embodiment 26, further comprising identifying, by the computing device, an injury or potential injury along a motor pathway associated with the one or more muscles.

Embodiment 28: The method of embodiment 27, further comprising forwarding, by the computing device to a display, an alert associated with an identified injury or potential injury.

Embodiment 29: The method of any combination of embodiments 27-28, further comprising generating a recommended action to ameliorate the identified injury or potential injury.

Embodiment 30: The method of any combination of embodiments 26-29, further comprising forwarding, by the computing device to a display, one or more of: the resulting muscle response electrical waveforms and an anatomical diagram depicting a location of the detected change in the resulting muscle response electrical waveforms.

Embodiment 31: The method of any combination of embodiments 26-30, further comprising: obtaining information from one or more of: an anesthesia machine, a neuromuscular junction testing machine, an oximetry machine, and a blood pressure machine; and determining, based on the information, whether the changes in the resulting muscle response electrical waveforms are due to one or more of: anesthesia, a paralytic effect, blood pressure, and perfusion changes.

Embodiment 32: The method of any combination of embodiments 26-31, further comprising averaging multiple resulting muscle response electrical waveforms obtained in response to performing multiple stimulations of the at least one cathodal electrode with the at least one anodal electrode to reduce noise.

Embodiment 33: The method of any combination of embodiments 1-32, further comprising receiving a user input indicating an accuracy of the resulting muscle response electrical waveforms.

Embodiment 34: The method of any combination of embodiments 1-33, wherein the patient is in a prone, supine, or lateral position.

Embodiment 35: The method of any combination of embodiments 1-34, wherein the surgery comprises a lumbar surgery or a cervical surgery.

Embodiment 36: An apparatus for monitoring reflexive motor responses in a patient during a surgery on the patient, the apparatus comprising: a memory comprising computer-executable instructions; and at least one processor configured to execute the computer-executable instructions and cause the apparatus to: stimulate a thoracic spinal region of the back of the patient with one or more pulses provided by at least one cathodal electrode positioned near the thoracic spinal region and at least one anodal electrode positioned over at least one anterior superior iliac crest area of the patient, the one or more pulses having a duration of at least 500 μs; and obtain, from at least one recording electrode on at least one leg of the patient, one or more resulting muscle response electrical waveforms from one or more muscles of one or more lower extremities of the patient.

Embodiment 37: A system for monitoring reflexive motor responses in a patient during a surgery on the patient, the system comprising: one or more cathodal electrodes positioned over a thoracic spinal region of the back of the patient; one or more anodal electrodes positioned over at least one anterior superior iliac crest area of the patient; one or more recording electrodes positioned on at least one leg of the patient; and a computing device configured to: stimulate the thoracic spinal region with one or more pulses using at least one of the one or more cathodal electrodes with at least one of the one or more anodal electrodes, wherein the one or more pulses have a duration of at least 500 μs; and obtain, from at least one of the one or more recording electrodes, one or more resulting muscle response electrical waveforms from one or more muscles of one or more lower extremities of the patient.

Embodiment 38: A method of monitoring reflexive motor responses in a patient during a surgery on the patient, the method comprising: stimulating with one or more pulses a high thoracic or low cervical spinal region of the back of the patient using at least one cathodal electrode positioned thereon and at least one anodal electrode positioned over at least one temporal bone of the patient, where the one or more pulses has a duration of at least 500 μs; and detecting, with at least one recording electrode on one or more upper extremities of the patient, one or more resulting muscle response electrical waveforms from one or more muscles.

Embodiment 39: The method of embodiment 38, wherein stimulating the at least one cathodal electrode comprises stimulating the at least one cathodal electrode cutaneously.

Embodiment 40: The method of any combination of embodiments 38-39, wherein stimulating the at least one cathodal electrode comprises stimulating the at least one cathodal electrode subcutaneously.

Embodiment 41: The method of any combination of embodiments 38-40, wherein the at least one cathodal electrode comprises a pair of cathodal electrodes located at the high thoracic or low cervical spinal region of the patient.

Embodiment 42: The method of any combination of embodiments 38-41, further comprising at least one cathodal electrode located at or between the tenth, eleventh, or twelfth thoracic vertebrae of the patient.

Embodiment 43: The method of any combination of embodiments 38-42, wherein the at least one cathodal electrode comprises a first pair of cathodal electrodes located on opposite sides of the thoracic spinal region of the back of the patient.

Embodiment 44: The method of embodiment 43, wherein the at least one cathodal electrode further comprises a second pair of cathodal electrodes located on opposite sides of the thoracic spinal region of the back of the patient above or below the first pair of cathodal electrodes.

Embodiment 45: The method of embodiment 44, wherein the stimulating the at least one cathodal electrode comprises alternating between stimulating the first pair of cathodal electrodes and the second pair of cathodal electrodes.

Embodiment 46: The method of any combination of embodiments 38-45, further comprising: determining, by a computing device in communication with the at least one recording electrode, the at least one cathodal electrode to stimulate, from a plurality of cathodal electrodes.

Embodiment 47: The method of embodiment 46, wherein determining the at least one cathodal electrode to stimulate comprises determining the at least one cathodal electrode to stimulate based on the one or more muscles to be monitored.

Embodiment 48: The method of any combination of embodiments 38-47, wherein the at least one anodal electrode comprises a pair of anodal electrodes positioned over each temporal bone of the patient.

Embodiment 49: The method of any combination of embodiments 38-48, further comprising a pair of anodal electrodes positioned over both anterior superior iliac crest areas of the patient.

Embodiment 50: The method of any combination of embodiments 38-49, wherein stimulating at least one cathodal electrode with the at least one anodal electrode comprises alternating stimulating with a first anodal electrode of a pair of anodal electrodes and a second anodal electrode of the pair of anodal electrodes.

Embodiment 51: The method of any combination of embodiments 38-50, wherein the at least one recording electrode is coupled to at least one muscle of the arm of the patient.

Embodiment 52: The method of any combination of embodiments 38-50, wherein the at least one recording electrode is coupled near an ulnar nerve of the patient.

Embodiment 53: The method of any combination of embodiments 38-52, further comprising: prior to the stimulating the at least one cathodal electrode with the at least one anodal electrode, stimulating one or more extremities of the patient using one or more peripheral electrodes located on the one or more extremities.

Embodiment 54: The method of embodiment 53, further comprising selecting the one or more peripheral electrodes to stimulate to enhance the one or more resulting muscle response electrical waveforms from the one or more muscles of the one or more extremities of the patient.

Embodiment 55: The method of any combination of embodiments 53-54, wherein the stimulating using the at least one cathodal electrode is performed after an inter-stimulus interval (ISI) after the stimulating using the one or more peripheral electrodes.

Embodiment 56: The method of any combination of embodiments 38-55, further comprising: determining, by a computing device in communication with the at least one recording electrode, the inadequacy of one or more resulting muscle response electrical waveforms from the one or more muscles of the one or more extremities of the patient; and stimulating the one or more lower of the patient using one or more peripheral electrodes in response to the determination.

Embodiment 57: The method of any combination of embodiments 53-56, further comprising determining, by the computing device, one or more of: a number of priming pulses, a duration of the priming pulses, a frequency of the priming pulses, and an intensity of the priming pulses used to stimulate the one or more peripheral electrodes.

Embodiment 58: The method of any combination of embodiments 38-57, further comprising determining, by a computing device in communication with the at least one recording electrode, one or more of: a number of pulses, a duration of the pulses, a frequency of the pulses, and an intensity of the pulses used to stimulate using the at least one cathodal electrode with the at least one anodal electrode.

Embodiment 59: The method of embodiment 58, wherein the determining is based on a target latency and amplitude of the one or more components of the resulting muscle response electrical waveforms in response to the stimulating.

Embodiment 60: The method of embodiment 59, wherein the determining comprises: using a first intensity of the pulses used to stimulate using the at least one cathodal electrode with the at least one anodal electrode; when a first latency and amplitude of a first one or more resulting muscle response electrical waveforms does not satisfy the target latency and amplitude, using a second intensity of the pulses used to stimulate the at least one cathodal electrode with the at least one anodal electrode, wherein the second intensity is higher than the first intensity; and when the first latency and amplitude of the first one or more resulting components of the muscle response electrical waveforms satisfies the target latency and amplitude, using the first intensity of the pulses used to stimulate the at least one cathodal electrode with the at least one anodal electrode.

Embodiment 61: The method of any combination of embodiments 38-60, further comprising: determining pre-operatively, by a computing device in communication with the at least one recording electrode, a first stimulation pattern to be used during the surgery; and determining, during the surgery, a second stimulation pattern to be used during the surgery.

Embodiment 62: The method of any combination of embodiments 38-61, further comprising receiving input from a user to perform the stimulating the at least one cathodal electrode with the at least one anodal electrode, wherein the stimulating is in response to the user input.

Embodiment 63: The method of any combination of embodiments 38-62, further comprising detecting, by a computing device in communication with the at least one recording electrode, a change in the resulting muscle response electrical waveforms and their components over time or compared to a baseline established pre-operatively, wherein the change comprises one or more of: a changed latency, amplitude, and morphology of the resulting muscle response electrical waveforms and their components.

Embodiment 64: The method of embodiment 63, further comprising identifying, by the computing device, an injury or potential injury along a motor pathway associated with the one or more muscles.

Embodiment 65: The method of embodiment 64, further comprising forwarding, by the computing device to a display, an alert associated with the identified injury or potential injury.

Embodiment 66: The method of any combination of embodiments 64-65, further comprising generating a recommended action to ameliorate the identified injury or potential injury.

Embodiment 67: The method of any combination of embodiments 63-66, further comprising forwarding, by the computing device to a display, one or more of: the resulting muscle response electrical waveforms and an anatomical diagram depicting a location of the detected change in the resulting muscle response electrical waveforms.

Embodiment 68: The method of any combination of embodiments 63-67, further comprising: obtaining information from one or more of: an anesthesia machine, a neuromuscular junction testing machine, an oximetry machine, and a blood pressure machine; and determining, based on the information, whether the changes in the resulting muscle response electrical waveforms are due to one or more of: anesthesia, a paralytic effect, blood pressure, and perfusion changes.

Embodiment 69: The method of any combination of embodiments 63-68, further comprising averaging multiple resulting muscle response electrical waveforms obtained in response to performing multiple stimulations of the at least one cathodal electrode with the at least one anodal electrode to reduce noise.

Embodiment 70: The method of any combination of embodiments 38-69, further comprising receiving a user input indicating an accuracy of the resulting muscle response electrical waveforms.

Embodiment 71: The method of any combination of embodiments 38-70, wherein the patient is in a prone, supine, or lateral position.

Embodiment 72: The method of any combination of embodiments 38-71, wherein the surgery comprises a lumbar surgery.

Embodiment 73: The method of any combination of embodiments 38-72, wherein the high thoracic or low cervical spinal region of the back of the patient comprises a region at or between the C5-T3 vertebrae of the patient.

Embodiment 74: The method of any combination of embodiments 38-73, wherein the one or more upper extremities of the patient comprises at least one of the arms, wrists, or region near the ulnar nerve of the patient.

Embodiment 75: The method of any combination of embodiments 38-74, wherein the at least one anodal electrode is positioned over a mastoid process of the temporal bone of the patient.

Embodiment 76: An apparatus for monitoring reflexive motor responses in a patient during a surgery on the patient, the apparatus comprising: a memory comprising computer-executable instructions; and at least one processor configured to execute the computer-executable instructions and cause the apparatus to: stimulate a high thoracic or low cervical spinal region of the back of the patient with one or more pulses provided by at least one cathodal electrode positioned near the cervical spinal region and at least one anodal electrode positioned over at least temporal bone of the patient, the one or more pulses having a duration of at least 500 μs; and obtain, from at least one recording electrode on one or more upper extremities of the patient, one or more resulting muscle response electrical waveforms from one or more muscles of one or more extremities of the patient.

Embodiment 77: A system for monitoring reflexive motor responses in a patient during a surgery on the patient, the system comprising: one or more cathodal electrodes positioned over a high thoracic or low cervical spinal region of the back of the patient; one or more anodal electrodes positioned over at least temporal bone of the patient; one or more recording electrodes positioned on one or more upper extremities of the patient; and a computing device configured to: stimulate the cervical spinal region with one or more pulses using at least one of the one or more cathodal electrodes with at least one of the one or more anodal electrodes, wherein the one or more pulses have a duration of at least 500 μs; and obtain, from at least one of the one or more recording electrodes, one or more resulting muscle response electrical waveforms from one or more muscles of one or more extremities of the patient.

### ADDITIONAL CONSIDERATIONS

The preceding description is provided to enable any person skilled in the art to practice the various aspects described herein. The examples discussed herein are not limiting of the scope, applicability, or aspects set forth in the claims. While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

Some of the aspects disclosed herein may have been disclosed in relation to a particular approach (e.g., lateral); however, other approaches (e.g., anterior, posterior, transforaminal, etc.) are also contemplated.

The aspects disclosed herein (or any part(s) or function(s) thereof) may be implemented using hardware, software, firmware, or a combination thereof and may be implemented in one or more computer systems or other processing systems. In fact, one example aspects may be directed toward one or more computer systems capable of carrying out the functionality described herein

The various illustrative logical blocks, modules and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, a system on a chip (SoC), or any other such configuration.

As used herein, “a processor,” “at least one processor” or “one or more processors” generally refers to a single processor configured to perform one or multiple operations or multiple processors configured to collectively perform one or more operations. In the case of multiple processors, performance of the one or more operations could be divided amongst different processors, though one processor may perform multiple operations, and multiple processors could collectively perform a single operation. Similarly, “a memory,” “at least one memory” or “one or more memories” generally refers to a single memory configured to store data and/or instructions, multiple memories configured to collectively store data and/or instructions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the aspects of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one aspect, the terms “about” and “approximately” refer to numerical parameters within 10% of the indicated range.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the terms “a,” “an,” “the,” and similar referents used in the context of describing the aspects of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the aspects of the present disclosure and does not pose a limitation on the scope of the present disclosure. No language in the specification should be construed as indicating any non-claimed element essential to the practice of the aspects of the present disclosure.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

Groupings of alternative elements or aspects disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain aspects are described herein, including the best mode known to the author(s) of this disclosure for carrying out the aspects disclosed herein. Variations on these described aspects will become apparent to those of ordinary skill in the art upon reading the foregoing description. The author(s) expects skilled artisans to employ such variations as appropriate, and the author(s) intends for the aspects of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific aspects disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Aspects of this disclosure so claimed are inherently or expressly described and enabled herein.

Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of monitoring reflexive motor responses in a patient during a surgery on the patient, the method comprising:
stimulating with one or more pulses a thoracic spinal region of a back of the patient using at least one cathodal electrode positioned thereon and at least one anodal electrode positioned over at least one anterior superior iliac crest area of the patient, where the one or more pulses has a duration of at least 500 μs; and
detecting, with at least one recording electrode on at least one leg of the patient, one or more resulting muscle response electrical waveforms from one or more muscles of one or more lower extremities of the patient.

2. The method of claim 1, wherein stimulating the at least one cathodal electrode comprises stimulating the at least one cathodal electrode cutaneously.

3. The method of claim 1, wherein stimulating the at least one cathodal electrode comprises stimulating the at least one cathodal electrode subcutaneously.

4. The method of claim 1, wherein the at least one cathodal electrode is located at or between the tenth, eleventh, or twelfth thoracic vertebrae of the patient.

5. The method of claim 1, further comprising a pair of cathodal electrodes located at the high thoracic or low cervical spinal region of the patient.

6. The method of claim 1, wherein the at least one cathodal electrode comprises a first pair of cathodal electrodes located on opposite sides of the thoracic spinal region of the back of the patient.

7. The method of claim 6, wherein the at least one cathodal electrode further comprises a second pair of cathodal electrodes located on opposite sides of the thoracic spinal region of the back of the patient above or below the first pair of cathodal electrodes.

8. The method of claim 7, wherein the stimulating the at least one cathodal electrode comprises alternating between stimulating the first pair of cathodal electrodes and the second pair of cathodal electrodes.

9. The method of claim 1, further comprising:
determining, by a computing device in communication with the at least one recording electrode, the at least one cathodal electrode to stimulate, from a plurality of cathodal electrodes over the thoracic spinal region of the back of the patient.

10. The method of claim 9, wherein determining the at least one cathodal electrode to stimulate comprises determining the at least one cathodal electrode to stimulate based on the one or more muscles to be monitored.

11. The method of claim 1, further comprising a pair of anodal electrodes positioned over a cranium of the patient.

12. The method of claim 1, wherein the pair of anodal electrodes are positioned over both temporal bones of a cranium of the patient.

13. The method of claim 1, wherein the at least one anodal electrode comprises a pair of anodal electrodes positioned over both anterior superior iliac crest areas of the patient.

14. The method of claim 1, wherein stimulating at least one cathodal electrode with the at least one anodal electrode comprises alternating stimulating with a first anodal electrode of a pair of anodal electrodes and a second anodal electrode of the pair of anodal electrodes.

15. The method of claim 1, wherein the at least one recording electrode is coupled to at least one muscle of the one or more lower extremities of the patient.

16. The method of claim 1, further comprising:
prior to the stimulating the at least one cathodal electrode with the at least one anodal electrode, stimulating one or more lower extremities of the patient using one or more peripheral electrodes located on the one or more lower extremities.

17. The method of claim 16, further comprising selecting the one or more peripheral electrodes to stimulate to enhance the one or more resulting muscle response electrical waveforms from the one or more muscles of the one or more lower extremities of the patient.

18. The method of claim 1, further comprising:
determining, by a computing device in communication with the at least one recording electrode, an inadequacy of one or more resulting muscle response electrical waveforms from the one or more muscles of the one or more lower extremities of the patient; and
stimulating the one or more lower extremities of the patient using one or more peripheral electrodes in response to the determination.

19. An apparatus for monitoring reflexive motor responses in a patient during a surgery on the patient, the apparatus comprising:
a memory comprising computer-executable instructions; and
at least one processor configured to execute the computer-executable instructions and cause the apparatus to:
stimulate a thoracic spinal region of a back of the patient with one or more pulses provided by at least one cathodal electrode positioned near the thoracic spinal region and at least one anodal electrode positioned over at least one anterior superior iliac crest area of the patient, the one or more pulses having a duration of at least 500 μs; and
obtain, from at least one recording electrode on at least one leg of the patient, one or more resulting muscle response electrical waveforms from one or more muscles of one or more lower extremities of the patient.

20. A system for monitoring reflexive motor responses in a patient during a surgery on the patient, the system comprising:
one or more cathodal electrodes positioned over a thoracic spinal region of a back of the patient;
one or more anodal electrodes positioned over at least one anterior superior iliac crest area of the patient;
one or more recording electrodes positioned on at least one leg of the patient; and
a computing device configured to:
stimulate the thoracic spinal region with one or more pulses using at least one of the one or more cathodal electrodes with at least one of the one or more anodal electrodes, wherein the one or more pulses have a duration of at least 500 μs; and
obtain, from at least one of the one or more recording electrodes, one or more resulting muscle response electrical waveforms from one or more muscles of one or more lower extremities of the patient.
